(19) United States
(12) Patent Application Publication
(10) Pub. No.: US 2026/0232972 A1
(43) Pub. Date: Aug. 13, 2026

(54) REINFORCEMENT WIRES FOR TENECTEPLASE DRUG-COATED BALLOON

(71) Applicant: UNIVERSITY OF KANSAS, Lawrence, KS (US)

(72) Inventors: Michael G. ABRAHAM, Kansas City, MO (US); Ian BALTZ, Olathe, KS (US)

(21) Appl. No.: 19/538,431

(22) Filed: Feb. 12, 2026

Related U.S. Application Data
(60) Provisional application No. 63/758,271, filed on Feb. 13, 2025.

(51) Int. Cl. A61M 25/10; A61B 17/22; A61F 2/82; A61F 2/958

(52) U.S. Cl. CPC A61M 25/1029; A61B 17/22; A61F 2/82; A61F 2/958; A61M 25/10; A61M 25/1002; A61M 2025/1031

(57) ABSTRACT

A drug-coated balloon for intracranial atherosclerosis. The drug-coated balloon includes a body portion having a distal end, a proximal end opposite the distal end, and a sidewall extending between the distal end and the proximal end. The body portion is movable between a deflated position and an inflated position. The drug-coated balloon also includes a plurality of wires supported within the body portion. Each wire restricts a portion of the balloon from expanding when the body portion moves to the inflated position. The portion of the balloon that is restricted from expanding forms recessed portions in the sidewall of the balloon. The recessed portions are configured to reduce an amount of plaque that moves from the main intracranial artery to perforator arteries.

### CROSS REFERENCE

This application claims the priority benefit of U.S. Provisional Patent Application No. 63/758,271 filed February 13, 2025, which is hereby incorporated herein by reference in its entirety.

### BACKGROUND

Atherosclerosis is the accumulation of calcified plaque in arteries that can lead to stenosis and potential occlusion (e.g., blockage) of an artery. Currently, there are drug-coated balloons that are used to reduce and prevent the risk of intimal hyperplasia after placing a stent in an artery. Intimal hyperplasia occurs after a stent is placed, where the atherosclerosis continues to grow and results in in-stent stenosis. Currently available drug-coated balloons are typically coated with paclitaxel, an agent that prevents intimal hyperplasia.

When patients present with a large vessel occlusion stroke, there is level 1A evidence for endovascular thrombectomy to remove the clot using aspiration catheters, stent-retrievers, or a combination of both. There are several etiologies of large vessel occlusion strokes: cardioembolic (e.g., clots from the heart), artery-to-artery emboli (e.g., clots from ruptured or breaking of an atherosclerotic plaque), and native atherosclerosis of the artery. The balloon described herein is typically used for native atherosclerosis.

When native atherosclerosis occurs, it can grow, resulting in progressive stenosis (e.g., narrowing) of the artery with eventual occlusion due to plaque and a combination of slow blood flow resulting in the formation of a blood clot (e.g., thrombosis). When a patient presents with large vessel occlusion due to native atherosclerosis, endovascular thrombectomy can be very difficult if not futile. Many times, the blocked artery can be opened, but high-grade residual stenosis is still present, which can lead to re-occlusion of the artery. Intracranial balloon angioplasty and stenting have been extensively studied. The results showed that balloon angioplasty and stenting had nearly a 15% worse outcome, including stroke. One of the main reasons that angioplasty and stenting are not as effective for intracranial atherosclerosis is that intracranial arteries have numerous perforator arteries arising from the parent artery (e.g., main artery). When balloon angioplasty and stenting are done, they can open the artery but have a high risk of causing a perforator artery stroke. Balloon angioplasty and stenting can also cause a snowplow effect where the plaque from the main artery is pushed into perforator arteries. Coronary arteries and the extracranial carotid arteries do not have perforator arteries, so the snowplow effect is not a concern when treating these arteries.

Therefore, there is a need in the art for a drug-coated balloon for intracranial atherosclerosis to reduce the snowplow effect of the plaque.

### SUMMARY

Various implementations described herein are related to a drug-coated balloon for intracranial atherosclerosis.

In some aspects, the disclosure is related to a drug-coated balloon for intracranial atherosclerosis. The drug-coated balloon is configured to be inserted through a portion of a main intracranial artery with plaque creating a progressive stenosis. The drug-coated balloon includes a body portion having a distal end, a proximal end opposite the distal end, and a sidewall extending between the distal end and the proximal end. The body portion is movable between a deflated position and an inflated position. The balloon also includes a plurality of wires supported within the body portion. Each wire restricts a portion of the balloon from expanding when the body portion moves to the inflated position. The portion of the balloon that is restricted from expanding forming recessed portions in the sidewall of the balloon. The recessed portions are configured to reduce an amount of plaque that moves from the main intracranial artery to perforator arteries.

In some aspects, the wires are longitudinal wires that extend longitudinally along a longitudinal axis of the body portion at least partially between the distal end and the proximal end.

In some aspects, the longitudinal wires are spaced from each other along an imaginary circle extending through the longitudinal wires, and the imaginary circle is positioned transverse to the longitudinal axis of the body portion.

In some aspects, the wires are equally spaced from each other along a circumference of the imaginary circle.

In some aspects, each longitudinal wire is spaced apart from each other by a distance, and the distance between adjacent longitudinal wires is in a range from 1 millimeter to 3 millimeters.

In some aspects, the longitudinal wires reinforce selected longitudinal portions of the balloon to restrict the selected longitudinal portions of the balloon from expanding, and the recessed portions are recessed longitudinal portions that extend at least partially between the distal end and the proximal end.

In some aspects, the wires form concentric rings that are spaced from each other along a longitudinal axis of the body portion. The wires reinforce selected circumferential portions of the balloon to restrict inflation of the selected circumferential portions of the balloon. The recessed portions are recessed circumference portions extending about a circumference of the balloon.

In some aspects, the plurality of wires include wires defining concentric rings that are spaced from each other along a longitudinal axis of the body portion and longitudinal wires that extend longitudinally along the longitudinal axis at least partially between the distal end and the proximal end.

In some aspects, the longitudinal wires reinforce selected longitudinal portions of the balloon to restrict the selected longitudinal portions of the balloon from expanding, the wires reinforce selected circumferential portions of the balloon to restrict inflation of the selected circumferential portions of the balloon, and the recessed portions include recessed circumference portions extending about a circumference of the balloon and recessed longitudinal portions that extend at least partially between the distal end and the proximal end.

In some aspects, the body portion of the balloon has an elongated oval geometry.

In some aspects, the distal end defines a rounded distal tip and the proximal end defines a rounded proximal tip, the sidewall includes an upper side wall and a lower side wall, and the sidewall extends between the rounded distal tip and the rounded proximal tip.

In some aspects, the upper side wall and the lower side wall each extend substantially parallel to each other.

In some aspects, the sidewall of the body portion includes a matrix coating having tenecteplase.

In some aspects, the balloon coupled is a hypotube. The wires each have opposing ends coupled to the hypotube. The wires each have an arcuate shape with a peak positioned between the ends, and the wires overlap each other to form a flower-like geometry.

In some aspects, the peak of each wire reinforces selected circumferential portions of the balloon to restrict inflation of the selected circumferential portions of the balloon. The recessed portions are recessed circumferential portions. The recessed circumferential portions extend about a circumference of the balloon.

In some aspects, the disclosure is related to a drug-coated balloon for intracranial atherosclerosis. The drug-coated balloon is configured to be inserted through a portion of a main intracranial artery with plaque creating a progressive stenosis. The drug-coated balloon includes a body portion having a distal end, a proximal end opposite the distal end, and a sidewall extending between the distal end and the proximal end. The body portion is movable between a deflated position and an inflated position. The balloon also includes a plurality of wires supported within the body portion. The wires defining concentric rings that are spaced from each other along a longitudinal axis of the body portion and longitudinal wires that extend longitudinally along the longitudinal axis at least partially between the distal end and the proximal end. Each wire restricting a portion of the balloon from expanding when the body portion moves to the inflated position. The portion of the balloon that is restricted from expanding forms recessed circumferential portions and recessed longitudinal portions in the sidewall of the balloon. The recessed circumferential portions and the recessed longitudinal portions are configured to reduce an amount of plaque that moves from the main intracranial artery to perforator arteries.

In some aspects, the longitudinal wires reinforce selected longitudinal portions of the balloon to restrict the selected longitudinal portions of the balloon from expanding, the wires reinforce selected circumferential portions of the balloon to restrict inflation of the selected circumferential portions of the balloon, and the recessed portions include recessed circumferential portions extending about a circumference of the balloon and recessed longitudinal portions that extend at least partially between the distal end and the proximal end.

In some aspects, the body portion of the balloon has an elongated oval geometry.

In some aspects, the techniques described herein relate to a drug-coated balloon, wherein: the longitudinal wires are spaced from each other along an imaginary circle extending through the longitudinal wires, and the imaginary circle is positioned transverse to the longitudinal axis of the body portion.

In some aspects, the techniques described herein relate to a drug-coated balloon, wherein the wires are equally spaced from each other along a circumference of the imaginary circle.

### BRIEF DESCRIPTION OF DRAWINGS

Various implementations of devices, systems, and methods are explained in even greater detail in the following drawings. The drawings are merely exemplary and certain features may be used singularly or in combination with other features. The drawings are not necessarily drawn to scale.

FIG. 1 is a side view of a patient illustrating plaque buildup in an intracranial artery.

FIG. 2A is a side view of a main intracranial artery with a section of the artery removed to illustrate the plaque buildup in the main intracranial artery and perforator arteries extending from the main intracranial artery.

FIG. 2B is a side view of the intracranial artery with a stent positioned within the intracranial artery causing a snowplow effect of the plaque into the perforator arteries.

FIG. 3 is a perspective view of the main intracranial artery illustrating a drug-coated balloon inserted therein.

FIG. 4 is a series of perspective views of the main intracranial artery illustrating insertion of the drug-coated balloon and stent therein.

FIG. 5 is a cross-sectional view of the drug-coated balloon, illustrating layers of a side wall of the drug-coated balloon.

FIG. 6A is a side cross-section view of the main intracranial artery with plaque causing progressive stenosis of the artery and a drug-coated balloon of the prior art in a deflated position and extending through a portion of the artery with progressive stenosis.

FIG. 6B is a side cross-section view of the main intracranial artery illustrating plaque causing progressive stenosis of the artery and the drug-coated balloon of FIG. 6A in an inflated position within the portion of the artery with progressive stenosis.

FIG. 7A is a side cross-section view of the main intracranial artery with plaque causing progressive stenosis of the artery and a drug-coated balloon according to an implementation of the disclosure in a deflated position and extending through a portion of the artery with progressive stenosis.

FIG. 7B is a side cross-section view of the main intracranial artery with plaque causing progressive stenosis of the artery and the drug-coated balloon of FIG. 7A in an inflated position within the portion of the artery with progressive stenosis, resulting in a less pronounced snow-plow effect.

FIG. 8 is a side view of a drug-coated balloon according to an implementation of the disclosure, illustrating a plurality of longitudinally extending wires extending between a distal end and a proximal end of the balloon.

FIG. 9A is a cross-sectional view of the drug-coated balloon of FIG. 8, illustrating the balloon in a deflated position.

FIG. 9B is a cross-sectional view of the drug-coated balloon of FIG. 8, illustrating the balloon in an inflated position.

FIG. 10 is a side view of a drug-coated balloon according to an implementation of the disclosure, illustrating the balloon in a deflated position with a plurality of concentric wires.

FIG. 11 is a side view of the drug-coated balloon of FIG. 10, illustrating the balloon in an inflated position.

FIG. 12 is a side view of a drug-coated balloon according to an implementation of the disclosure, illustrating the balloon in a deflated position with a plurality of concentric wires.

FIG. 13 is a side view of the drug-coated balloon of FIG. 12, illustrating the balloon in an inflated position.

FIG. 14 is a side view of a drug-coated balloon according to an implementation of the disclosure, illustrating the balloon in a deflated position with a plurality of concentric wires and a plurality of longitudinally extending wires extending between the distal end and the proximal end.

FIG. 15A is a side view of the drug-coated balloon of FIG. 14, illustrating the balloon in an inflated position.

FIG. 15B is a cross-sectional view of the drug-coated balloon of FIG. 14, illustrating the balloon in the inflated position.

FIG. 16 is a front view of the drug-coated balloon according to another implementation of the disclosure.

FIG. 17 is a cross-sectional view of the drug-coated balloon of FIG. 16, illustrating the balloon in an inflated position.

### DETAILED DESCRIPTION

The devices, systems, and methods disclosed herein provide for a drug-coated balloon for intracranial atherosclerosis. The drug-coated balloon includes a plurality of wires configured to restrict selected portions of the balloon from expanding when the balloon is in an inflated position. The construction of the balloon reduces plaque from snowplowing from an intracranial artery into perforator arteries.

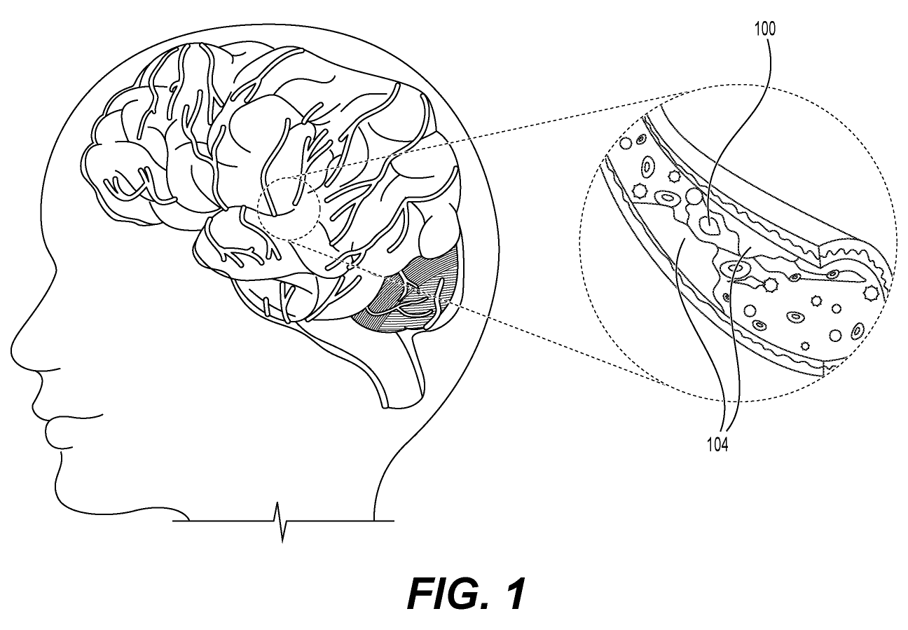

Now with reference to FIGS. 1-3, a main intracranial artery 100 having a plaque 104 neointima therein. For example, when native atherosclerosis occurs (FIG. 1), the plaque 104 causing the native atherosclerosis can grow, which results in progressive stenosis (e.g., narrowing) of the main intracranial artery 100. If surgical intervention is not performed, eventual occlusion of the main intracranial artery may occur due to the plaque 104 and a combination of slow blood flow, which can result in the formation of a blood clot (e.g., thrombosis). Further, the main intracranial artery 100 has perforator arteries 108 extending therefrom.

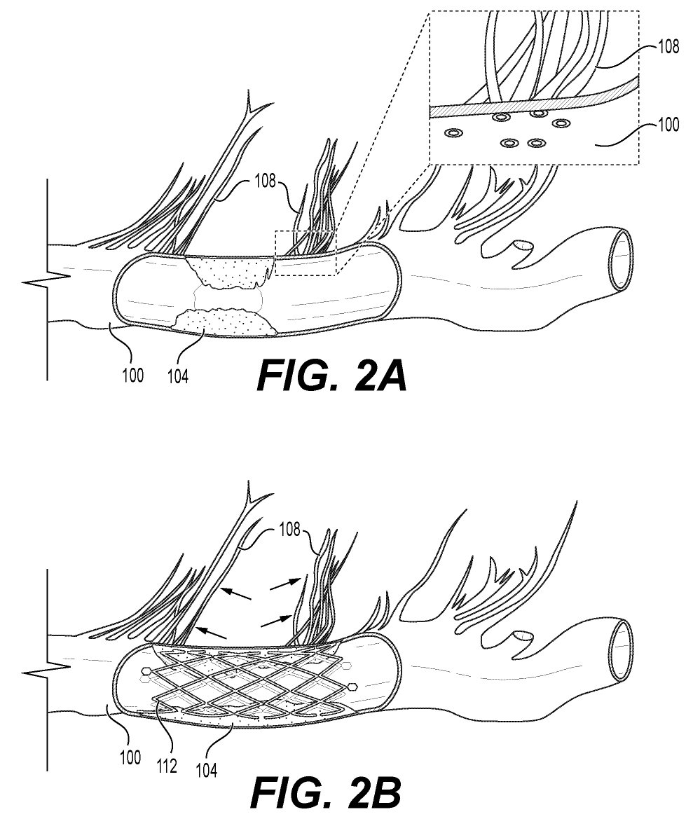

Drug-coated balloons, which are described in more detail below, can be used to open the main intracranial artery 100. Due to the construction of the intracranial arteries, there is a risk that plaque from the intracranial artery 100 can enter the perforator arteries 108. However, balloon angioplasty and stenting can also cause a snowplow effect where the plaque from the main artery is pushed into perforator arteries 108. In addition, another risk with placing an arterial stent 112 (FIG. 2B) within the intracranial artery 100 is hemorrhage. Patients who have the arterial stent 112 placed within the main intracranial artery 100 need to be on dual antiplatelet therapy because the stent 112 is considered a foreign body. Without dual antiplatelet therapy, the stent 112 can quickly thrombose. Therefore, dual antiplatelet therapy may include a combination of medications such as aspirin, clopidogrel, ticagrelor, and/or prasugrel. Since the medication requires multiple doses to reach a therapeutic level, the therapeutic drug levels can be reached by giving a loading dose of the dual antiplatelet therapy, which is typically 4-5 times the daily dose. When giving such a large dose of dual antiplatelet therapy in the acute setting, there is a risk of hemorrhagic conversion of the already stroked brain tissue.

Another risk with intracranial stenting is in-stent stenosis. Once the stent 112 is placed, a process called endothelialization occurs, where the normal artery tissue grows over the stent, so eventually the stent becomes “one” with the body. In some patients, endothelialization can be more extreme and result in re-stenosis of the stent 112, which can lead to recurrent symptoms and potentially retreatment. As such, the drug-coated balloon 300 described herein may be used to reduce re-stenosis of the stent and overcome a snowplow effect of plaque 104 into the perforator arteries 108.

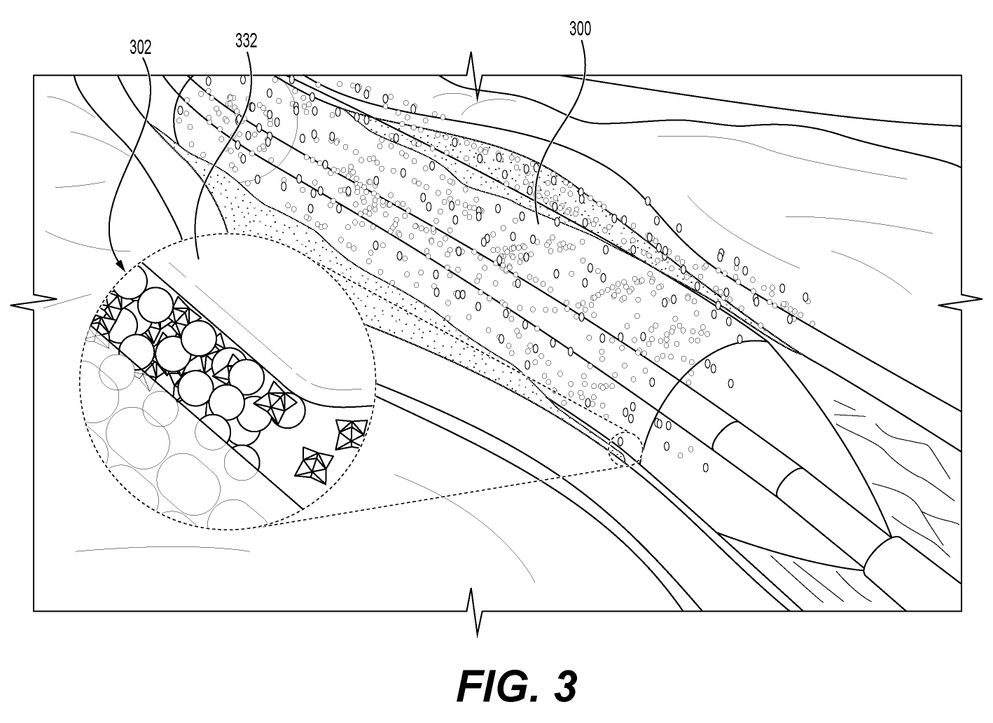

As illustrated in FIG. 3, the drug-coated balloon 300 includes a sidewall 332 and a matrix coating 302 on the sidewall 332. For example, the matrix coating 302 may comprise a drug such as tenecteplase (TNK) to aid with dissolving blood clots and paclitaxel to aid with inhibiting or reducing the proliferation of cells. The matrix coating facilitates use of the balloon 300 in non-acute settings due to atherosclerotic stenosis that results in recurrent mini-strokes. Paclitaxel, an antiproliferative drug, forms numerous decentralized and unorganized microtubules within the cytoplasm by shifting the microtubule equilibrium towards microtubule assembly. These alterations of the cytoskeleton interfere with many functions of the cell, such as proliferation, motility, migration, intracellular transport, and transmembrane signaling. Since several important biological processes (e.g., like the activation of protein kinases) are associated with microtubule depolymerization, these processes are therefore inhibited by paclitaxel.

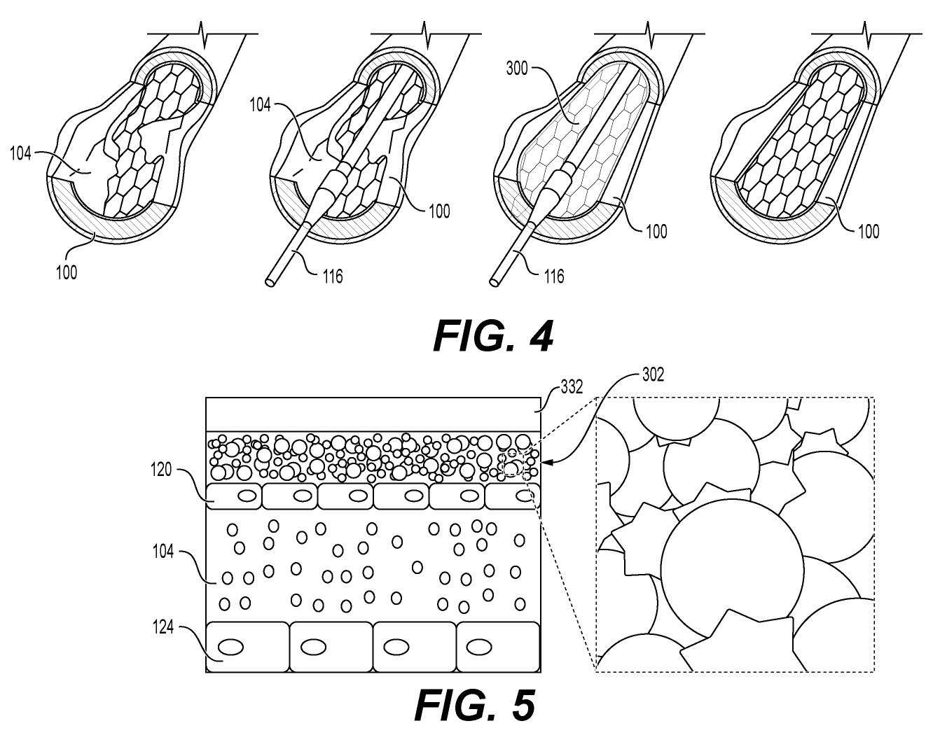

FIGS. 4 and 5 illustrate use of the balloon 300 as the balloon 300 is inserted within the main intracranial artery 100 over a guidewire 116. Further, the interaction between the matrix coating 302 and surfaces of the main intracranial artery 100. For example, the active drugs of the matrix coating may contact an endothelium 120 of the main intracranial artery 100. The active drugs may move through the endothelium 120 and interact with the plaque 104 or neointima to help break up the plaque 104. In some implementations, the active drugs may travel to the tunica media of the main intracranial artery 100.

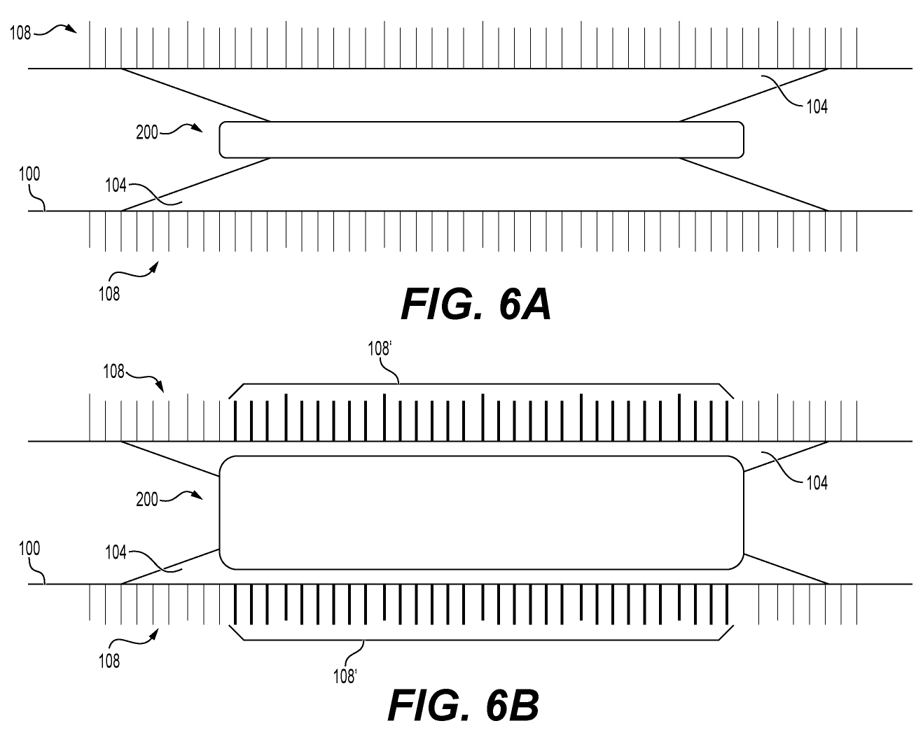

Now with reference to FIG. 6A and 6B, a balloon 200 according to prior art is illustrated within the main intracranial artery 100 having plaque 104 causing artery stenosis (e.g., narrowing). FIG. 6A illustrates the balloon 200 in a deflated position as the balloon 200 is inserted through a portion of the main intracranial artery 100 with the progressive stenosis. FIG. 6B illustrates the drug-coated balloon in an inflated position. Due to the construction of the balloon 200, the plaque 104 is pushed or snowplowed into a portion of the perforator arteries 108’ positioned adjacent to the plaque 104.

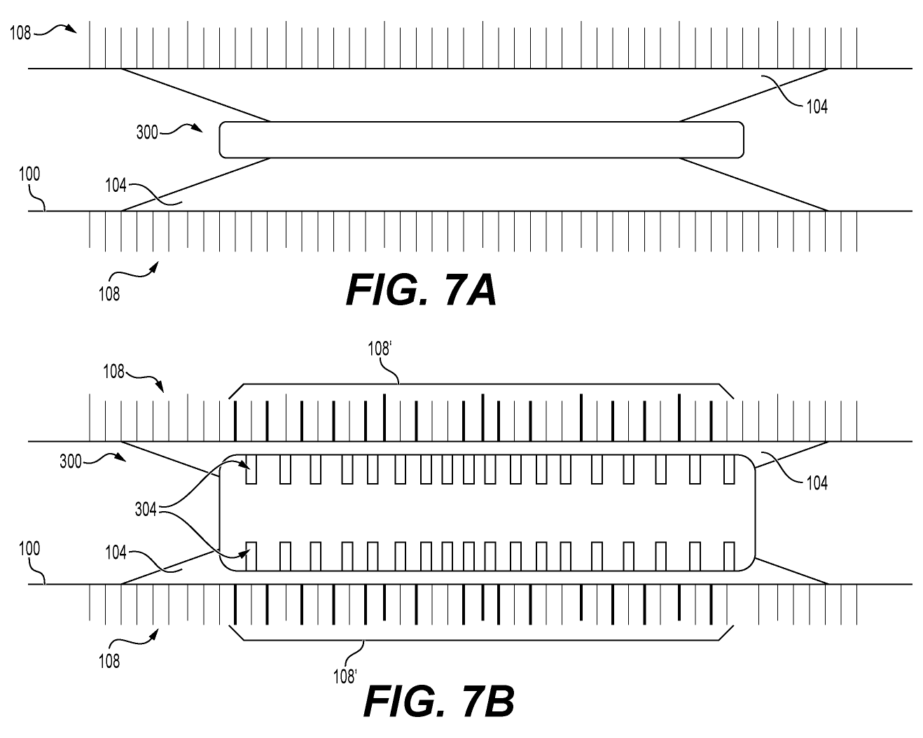

Now with reference to FIG. 7A and 7B, a drug-coated balloon 300 according to an implementation of the disclosure is illustrated within the main intracranial artery 100 having plaque 104 causing artery stenosis (e.g., narrowing). FIG. 7A illustrates the drug-coated balloon 300 in a deflated position as the balloon 300 is inserted through a portion of the main intracranial artery 100 with progressive stenosis. FIG. 7B illustrates the drug-coated balloon 300 in an inflated position. As described herein, the balloon 300, the construction of the balloon 300, and the other balloons described herein are configured to restrict sections of the balloon 300 from expanding when the balloon 300 is in the inflated position. In other words, a plurality of recessed portions 304 are formed in the inflated position. The recessed portions 304 are configured to reduce the amount of plaque 104 that is pushed through a portion of the perforator arteries 108’ positioned adjacent the plaque 104. In some implementations, the plaque 104 may move into the recessed portions 304 instead of snowplowing into the entire portion of the perforator arteries 108’ positioned adjacent the plaque 104.

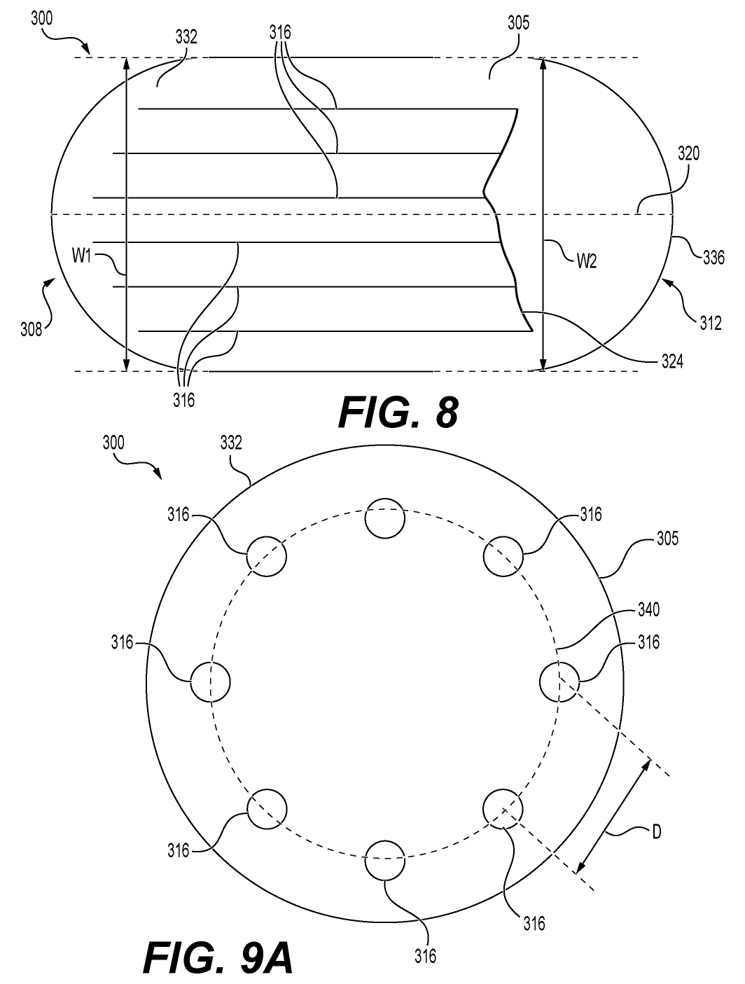

Now with reference to FIG. 8, the drug-coated balloon 300 is illustrated in detail. The balloon 300 includes a body portion 305 having a distal end 308 and a proximal end 312 opposite the distal end 308. A plurality of wires 316 are supported within the body portion 305 and extend between the distal end 308 and the proximal end 312. In the illustrated implementation, the wires 316 extend longitudinally (e.g., along a longitudinal axis 320) between the distal end 308 and the proximal end 312. In some implementations, the nitinol wires 316 may be operably coupled to an attachment structure 324. The attachment structure 324 facilitates uniform engagement of the wires 316 to restrict portions of the balloon 300 from expanding as the balloon 300 is moved towards the inflated position. In some implementations, the wires 316 are constructed of nitinol, chromium-cobalt-tungsten alloy, platinum, stainless steel. The construction of the wires 316 reinforces selected portions of the balloon 300 to restrict inflation of the selected portions of the balloon 300.

The body portion 305 of the balloon 300 has an elongated oval geometry, when viewed from the side (FIG. 8). Compared to other balloons of the prior art, which are generally uniform in shape, the elongated oval geometry of the balloon 300 facilitates insertion of the balloon 300 into the main intracranial artery 100. In the illustrated implementation, the body portion 305 tapers from the distal end 308 to the proximal end 312. For example, the distal end 308 may define a rounded distal tip 328 that terminates at a side wall 332a, 332b. The tapered sidewall comprises an upper side wall 332a and a lower side wall 332b when viewed from the side in FIG. 8. In some implementation, the upper and lower side walls 332a, 332b are substantially parallel to each other.

The balloon 300 has a first width W1 defined at the intersection of the rounded distal tip 328 and the tapered side wall 332. The proximal end 312 may define a rounded proximal tip 336 that terminates at the tapered side wall 332. The balloon 300 has a second width W2 defined at the intersection of the rounded proximal tip 336 and the tapered side wall 332. In some implementations, the width W1 may be the same as the width W2. In other implementations, the width W1 may be less than the width W2. In other words, the body portion 305 may have an increasing tapered profile from the distal end 308 to the proximal end 312. When the balloon 300 is in the deflated position the width W1 and the width W2 may be approximately 30 to 80 percent of a width of the main intracranial artery 100.

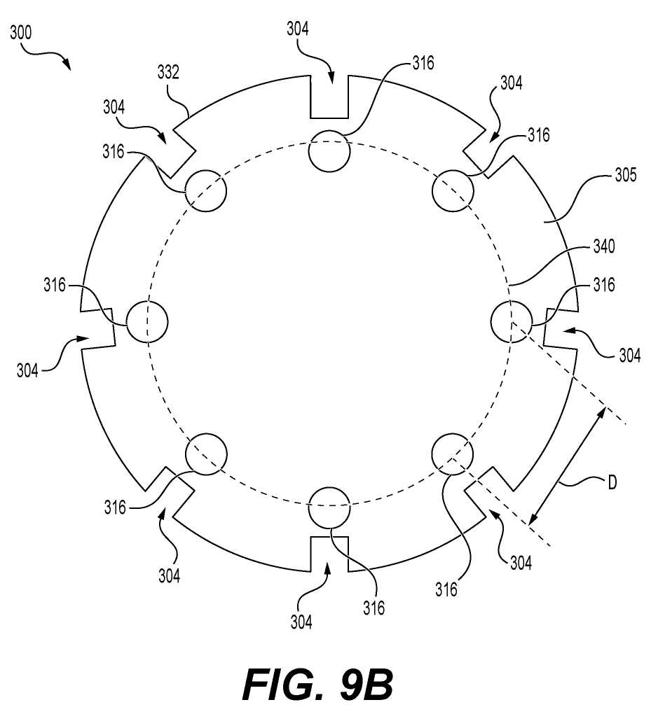

Now with reference to FIGS. 9A and 9B, movement of the balloon 300 between the deflated position (FIG. 9A) and the inflated position (FIG. 9B) is illustrated. The wires 316 are spaced from each other along an imaginary circle 340 extending through the wires 316. For example, the imaginary circle 340 is positioned transverse to the longitudinal axis (e.g., shown in the cross-sectional view of FIG. 9B). In the illustrated implementation, the balloon 300 comprises eight wires 316 that are each spaced apart from each other by a distance D. In other implementations, the balloon 300 may have more (e.g., nine, 10, etc.) or less (e.g., seven, six, etc.) wires 316. The distance D between adjacent wires 316 may be in a range from 1 millimeter to 3 millimeters. In other implementations, the distance D between each wire 316 may be different (e.g., the wires 316 may not be equally spaced).

As the balloon 300 is moved towards the expanded position (FIG. 9B), the wires 316 are configured to restrict longitudinal sections of the balloon 300 from expanding. The restriction of expansion along a path of the wires 316 creates the plurality of recessed longitudinal portions 304. As discussed herein, the positioning of the recessed longitudinal portions 304 reduces plaque from snowplowing from the intracranial artery 100 into perforator arteries 108. Further, it should be appreciated that the length of wires 316 (e.g., between the distal and proximal ends 308, 312) corresponds to the length of the recessed longitudinal portions 304. Therefore, in some implementations, the recessed longitudinal portions 304 may extend the entire length of the balloon 300 or a selected length of the balloon 300.

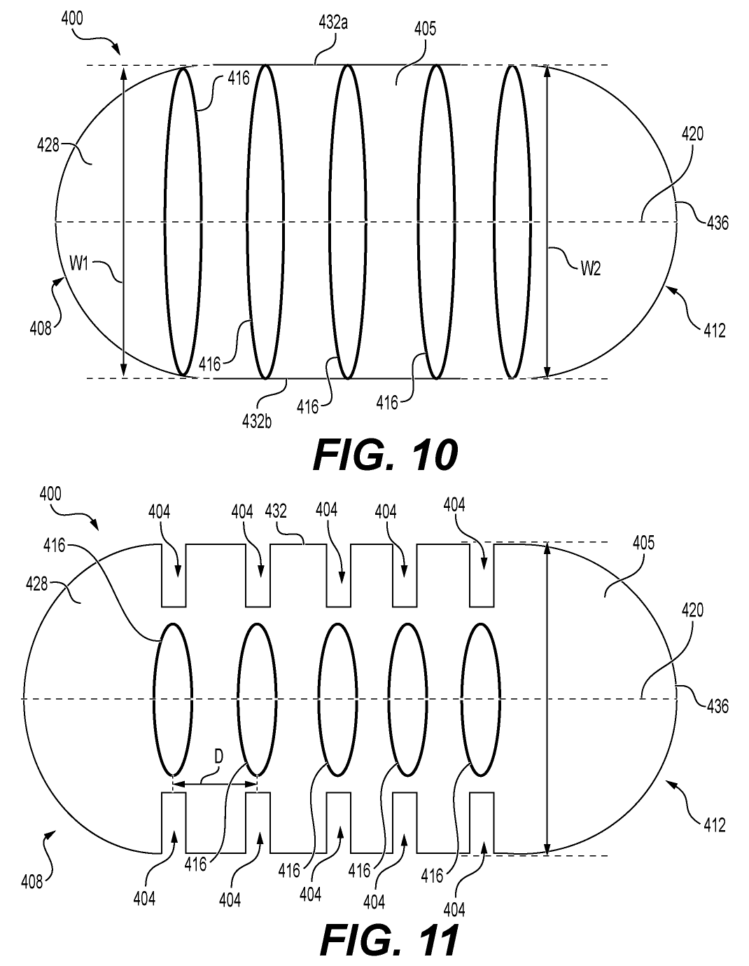

Now with reference to FIGS. 10 and 11, a drug-coated balloon 400 according to an alternative implementation is illustrated. The drug-coated balloon 400 is similar to the drug-coated balloon 300 described above with reference to FIGS. 7A-9, except as noted, and the following description focuses primarily on differences between the drug-coated balloon 400 and the drug-coated balloon 300. In addition, common features and elements of the drug-coated balloon 400 corresponding with features and elements of the drug-coated balloon 300 are given common reference numbers plus 100.

The balloon 400 includes a body portion 405 having a distal end 408 and a proximal end 412 opposite the distal end 408. A plurality of wires 416 are supported within the body portion 405 at positions between the distal end 408 and the proximal end 412. In the illustrated implementation, each wire 416 defines a concentric ring. For example, at selected longitudinal positions along a longitudinal axis 420, the balloon 400 comprises the concentric ring. Further, each concentric ring is spaced from an adjacent concentric ring along the longitudinal axis 420 of the balloon 400. In some implementations, the wires 416 may be operably coupled to each other (e.g., via an attachment structure) to facilitate uniform engagement of the wires 416 to restrict portions of the balloon 400 from expanding as the balloon 400 is moved towards an inflated position (FIG. 11). In some implementations, each concentric ring at the different longitudinal position along balloon have substantially the same diameter as the adjacent concentric ring. The positioning of the wires 416 reinforces selected circumferential portions of the balloon 400 to restrict inflation of the selected circumferential portions of the balloon 400.

The balloon 400 is movable between a deflated position (FIG. 10) and the inflated position (FIG. 11). The wires 416 are concentric rings at each longitudinal section of the balloon 400. Further, the concentric ring is spaced from an adjacent concentric ring by a distance D (FIG. 11) defined along the longitudinal axis 420. In the illustrated implementation, the balloon 400 comprises five concentric wires 416 that are each spaced from each by the distance D. In other implementations, the balloon 400 may have more (e.g., six, seven, etc.) or less (e.g., four, three, etc.) wires 416. The distance D may be in a range from 1 millimeter to 3 millimeters. In other implementations, the distance between each wire 416 may be different (e.g., the wires 416 may not be equally spaced). In some implementations, each wire 416 defining the concentric ring has substantially the same diameter as the adjacent concentric ring. For example, each wire 416 has a diameter that is equal to or less than a width W1 of the distal end 408 of the balloon 400. In other implementations, each wire 416 may have a diameter that corresponds to the width of the balloon 400 in the deflated position (FIG. 10).

As the balloon 400 is moved towards the expanded position (FIG. 11), the wires 416 are configured to restrict sections of the balloon 400 from expanding. In contrast to the balloon 300, which creates longitudinal sections (e.g., extending along the longitudinal axis), the concentric construction of the wires 416 facilitates formation of recessed circumference portions 404 (e.g., extending about a circumference of the balloon 400). The restriction of expansion along a circumference path of the wires 416 creates the plurality of recessed circumference portions 404. As discussed herein, the positioning of the recessed circumferential portions 404 reduces plaque from snowplowing from the intracranial artery 100 into perforator arteries 108. Further, it should be appreciated that a width of wires 416 or construction of the concentric ring may correspond to a width and/or geometry of the recessed circumferential portions 404. In other implementations, each wire 416 may be adjusted to form the desired width of the recessed portions.

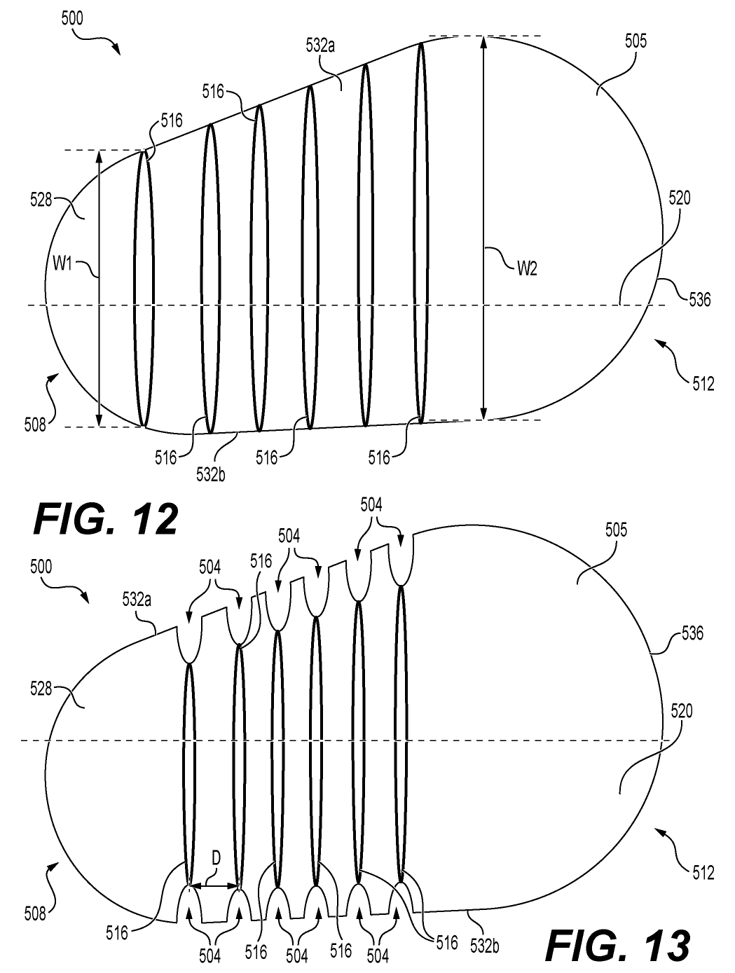

Now with reference to FIGS. 12 and 13, a drug-coated balloon 500 according to an alternative implementation is illustrated. The drug-coated balloon 500 is similar to the drug-coated balloons 300, 400 described above with reference to FIGS. 7A-11, except as noted, and the following description focuses primarily on differences between the drug-coated balloon 500 and the drug-coated balloons 300, 400. In addition, common features and elements of the drug-coated balloon 500 corresponding with features and elements of the drug-coated balloons 300, 400 are given common reference numbers plus 100 or 200.

The balloon 500 includes a body portion 505 having a distal end 508 and a proximal end 512 opposite the distal end 508. A plurality of wires 516 are supported within the body portion 505 at positions between the distal end 508 and the proximal end 512. In the illustrated implementation, each wire 516 defines a concentric ring. For example, at selected longitudinal positions along a longitudinal axis 520, the balloon 500 comprises the concentric ring. In some implementations, the wires 516 may be operably coupled to each other (e.g., via an attachment structure) to facilitate uniform engagement of the wires 516 to restrict portions of the balloon 500 from expanding as the balloon 500 is moved towards an inflated position (FIG. 13). The positioning of the wires 516 reinforces selected circumferential portions of the balloon 500 to restrict inflation of the selected circumferential portions of the balloon 500.

The balloon 500 is movable between a deflated position (FIG. 12) and the inflated position (FIG. 13). The wires 516 are concentric rings at each longitudinal section of the balloon 400. Further, the concentric ring is spaced from an adjacent concentric ring by a distance D (FIG. 13). In the illustrated implementation, the balloon 500 comprises six concentric wires 516 that are each spaced apart from each other by a distance D. In other implementations, the balloon 500 may have more (e.g., seven, etc.) or less (e.g., four, three, etc.) wires 516. The distance D may be in a range from 1 millimeter to 3 millimeters. In other implementations, the distance between each wire 516 defining the concentric ring may be different (e.g., the wires 516 may not be equally spaced). In the illustrated implementation, each wire 516 defining the concentric rings has an increasing diameter as the wires are positioned closer to the proximal end 512. For example, the wire 516 closest to the distal end 508 has the smallest diameter, and the wire 516 closest to the proximal end 512 has the largest diameter. In other words, each wire 516 may have a diameter that corresponds to the width of the balloon 500 in the deflated position (FIG. 12).

In the illustrated implementation, the balloon 500 tapers from the distal end 508 to the proximal end 512. For example, the distal end 508 may define a rounded distal tip 528 that terminates at a tapered side wall 532a, 532b. The tapered sidewall comprises an upper tapered side wall 532a and a lower tapered side wall 532b when viewed from the side in FIG. 12. In the illustrated implementation, the upper tapered side wall 532a may extend at a greater angle than an angle the lower tapered side wall 532b extends. In other words, the upper tapered side wall 532a is more tapered than the lower tapered side wall 532b. In some implementations, the lower tapered side wall may be relatively straight or have a slight taper.

As the balloon 500 is moved towards the expanded position (FIG. 13), the wires 516 configured to restrict sections of the balloon 500 from expanding. In contrast to the balloon 500, which creates longitudinal sections (e.g., extending along the longitudinal axis), the concentric construction of the wires 516 facilitates radial recessed portions (e.g., extending about a circumference of the balloon 500). The restriction of expansion along a circumference path of the wires 516 creates the plurality of recessed circumference portions 504. The positioning of the recessed circumferential portions 504 reduces plaque from snowplowing from the intracranial artery 100 into perforator arteries 108. Further, it should be appreciated that a width of wires 516 may correspond to a width of the recessed circumferential portions 504. In other implementations, each wire 516 may be adjusted to form the desired width of the recessed portions.

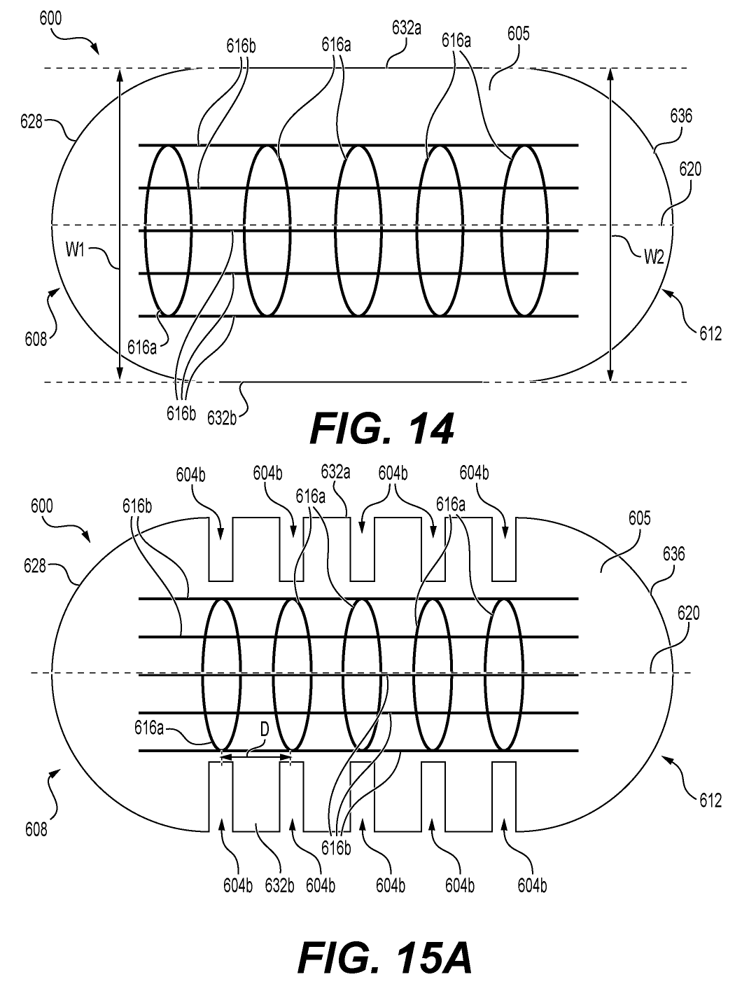

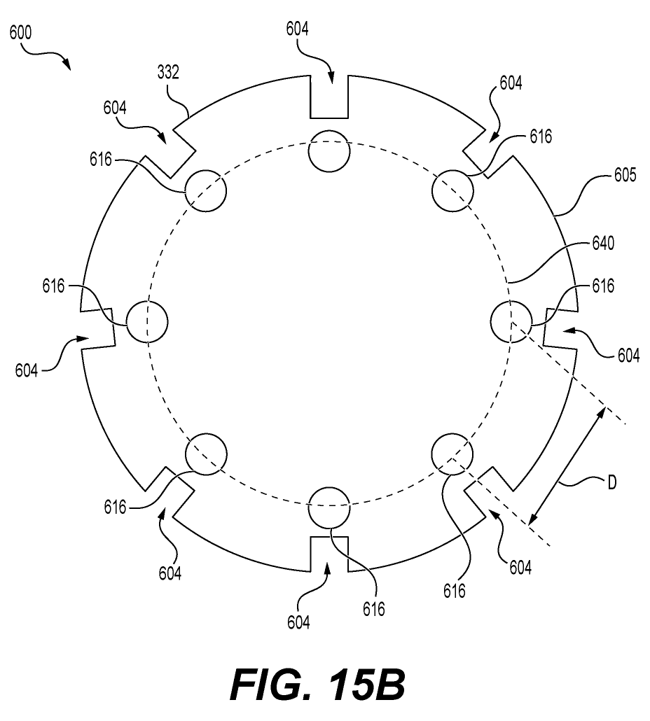

Now with reference to FIGS. 14 and 15, a drug-coated balloon 600 according to an alternative implementation is illustrated. The drug-coated balloon 600 is similar to the drug-coated balloons 300, 400, 500 described above with reference to FIGS. 7A-13, except as noted, and the following description focuses primarily on differences between the drug-coated balloon 600 and the drug-coated balloons 300, 400, 500. In addition, common features and elements of the drug-coated balloon 600 corresponding with features and elements of the drug-coated balloons 300, 400, 500 are given common reference numbers plus 100, 200, or 300.

The balloon 600 includes a body portion 605 having a distal end 608 and a proximal end 612 opposite the distal end 608. A plurality of wires 616a, 616b are supported within the body portion 605 at positions between the distal end 608 and the proximal end 612. In the illustrated implementation, the wires 616a, 616b comprise wires each defining concentric rings 616a. Further, the concentric ring is spaced from an adjacent concentric ring by a distance D (FIG. 11) defined along the longitudinal axis 620. Longitudinal wires 616b that extend longitudinally (e.g., along a longitudinal axis 620) between the distal end 608 and the proximal end 612. The longitudinal wires 616b are spaced from each other along an imaginary circle 640 (FIG. 15B) extending through the wires 616b. It should be appreciated that the description above related to the wires 316 applies equally to the wires 616b. For example, the longitudinal wires 616b restrict longitudinal sections of the balloon 600 from expanding when the balloon 600 is in the inflated position (FIG. 15B). In other words, a plurality of recessed longitudinal portions 604b are formed in the inflated position.

Further, the concentric rings 616a facilitate formation of circumferential recessed portions 604b (e.g., extending about a circumference of the balloon 600 in FIG. 15A) when the balloon 600 is in the inflated position. It should be appreciated that the description above related to the wires 416 applies equally to the concentric rings 616a. The positioning of the wires 616a, 616b reinforces selected longitudinal and circumferential portions of the balloon 600 to restrict inflation of the selected longitudinal portions of the balloon 600. The positioning of the recessed longitudinal portions 604b and the recessed circumferential portions 604b reduces plaque from snowplowing from the intracranial artery 100 into perforator arteries 108.

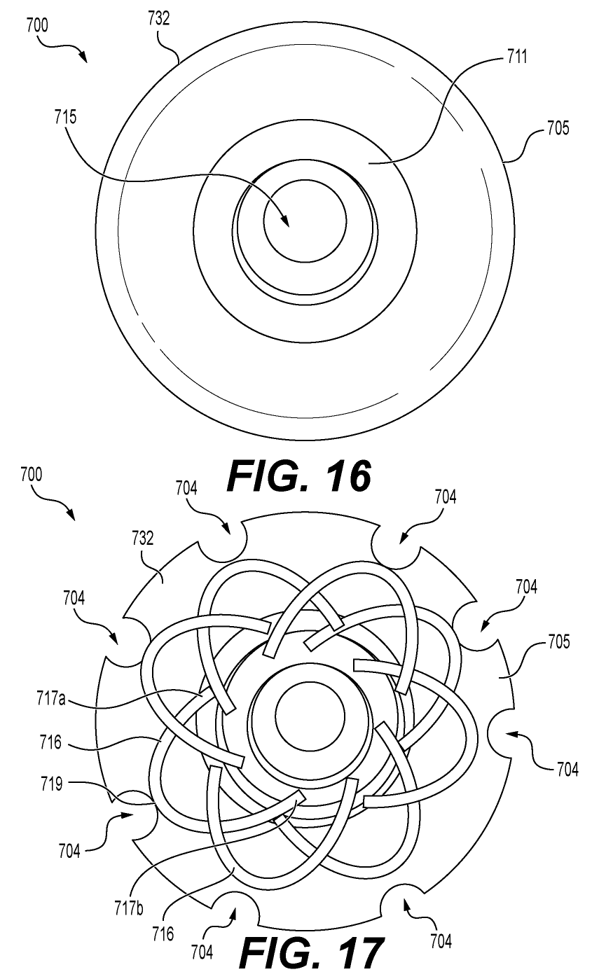

Now with reference to FIGS. 16 and 17, a drug-coated balloon 700 according to an alternative implementation is illustrated. The drug-coated balloon 700 is similar to the drug-coated balloons 300, 400, 500, 600 described above with reference to FIGS. 7A-15, except as noted, and the following description focuses primarily on differences between the drug-coated balloon 700 and the drug-coated balloons 300, 400, 500, 600. In addition, common features and elements of the drug-coated balloon 700 corresponding with features and elements of the drug-coated balloons 300, 400, 500, 600 are given common reference numbers plus 100, 200, 300, or 400.

It should be appreciated that for the sake of brevity, the geometry of a body portion 705 of the balloon 700 may be similar to any of the body portions 305 described above. As such, the body portion 705 has a distal end, a proximal end, and a sidewall 732 extending between. The balloon 700 is coupled to a hypotube 711 defining an aperture 715 extending therethrough. The aperture 715 may be sized to receive a device (e.g., a guidewire) or allow blood to flow therethrough. A plurality of wires 716 having opposing ends 717a, 717b coupled to the hypotube 711. The wires 716 have an arcuate shape with a peak 719 positioned between the ends 717a, 717b. The wires 716 overlap each other to form a flower-like geometry. As illustrated in FIG. 17, the peak 719 of each wire 716 facilitates formation of recessed circumference portions 704 (e.g., extending about a circumference of the balloon 700). The restriction of expansion along a circumference path of the wires 716 creates the plurality of recessed circumference portions 704. Similar to the concentric rings formed by the wires 416 (FIG. 11), the body portion may comprise a plurality of structures that are offset from each other along a longitudinal axis of the balloon 700.

Although the figures show a specific order of method steps, the order of the steps may differ from what is depicted. It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” or “substantially” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A drug-coated balloon for intracranial atherosclerosis, the drug-coated balloon configured to be inserted through a portion of a main intracranial artery with plaque creating a progressive stenosis, the drug-coated balloon comprising:
a body portion having a distal end, a proximal end opposite the distal end, and a sidewall extending between the distal end and the proximal end, the body portion movable between a deflated position and an inflated position; and
a plurality of wires supported within the body portion, each wire restricting a portion of the balloon from expanding when the body portion moves to the inflated position, the portion of the balloon that is restricted from expanding forming recessed portions in the sidewall of the balloon,
wherein the recessed portions are configured to reduce an amount of plaque that moves from the main intracranial artery to perforator arteries.

2. The drug-coated balloon of claim 1, wherein the wires are longitudinal wires that extend longitudinally along a longitudinal axis of the body portion at least partially between the distal end and the proximal end.

3. The drug-coated balloon of claim 2, wherein:
the longitudinal wires are spaced from each other along an imaginary circle extending through the longitudinal wires, and
the imaginary circle is positioned transverse to the longitudinal axis of the body portion.

4. The drug-coated balloon of claim 3, wherein the wires are equally spaced from each other along a circumference of the imaginary circle.

5. The drug-coated balloon of claim 4, wherein:
each longitudinal wire is spaced apart from each other by a distance, and
the distance between adjacent longitudinal wires is in a range from 1 millimeter to 3 millimeters.

6. The drug-coated balloon of claim 2, wherein:
the longitudinal wires reinforce selected longitudinal portions of the balloon to restrict the selected longitudinal portions of the balloon from expanding, and
the recessed portions are recessed longitudinal portions that extend at least partially between the distal end and the proximal end.

7. The drug-coated balloon of claim 1, wherein:
the wires form concentric rings,
the wires reinforce a selected circumferential portion of the balloon to restrict inflation of the selected circumferential portions of the balloon, and
the recessed portions are recessed circumference portions extending about a circumference of the balloon.

8. The drug-coated balloon of claim 1, wherein:
the plurality of wires comprise wires defining concentric rings and longitudinal wires that extend longitudinally along the longitudinal axis at least partially between the distal end and the proximal end.

9. The drug-coated balloon of claim 8, wherein:
the longitudinal wires reinforce selected longitudinal portions of the balloon to restrict the selected longitudinal portions of the balloon from expanding,
the wires reinforce selected circumferential portions of the balloon to restrict inflation of the selected circumferential portions of the balloon, and
the recessed portions comprise recessed circumference portions extending about a circumference of the balloon and recessed longitudinal portions that extend at least partially between the distal end and the proximal end.

10. The drug-coated balloon of claim 1, wherein the body portion of the balloon has an elongated oval geometry.

11. The drug-coated balloon of claim 10, wherein:
the distal end defines a rounded distal tip and the proximal end defines a rounded proximal tip,
the sidewall comprises an upper side wall and a lower side wall, and
the sidewall extends between the rounded distal tip and the rounded proximal tip.

12. The drug-coated balloon of claim 11, wherein the upper side wall and the lower side wall each extend substantially parallel to each other.

13. The drug-coated balloon of claim 1, wherein the sidewall of the body portion comprises a matrix coating having tenecteplase.

14. The drug-coated balloon of claim 1, wherein:
the balloon coupled is a hypotube,
the wires each have opposing ends coupled to the hypotube,
the wires each have an arcuate shape with a peak positioned between the ends, and
the wires overlap each other to form a flower-like geometry.

15. The drug-coated balloon of claim 14, wherein:
the peak of each wire reinforces selected circumferential portions of the balloon to restrict inflation of the selected circumferential portions of the balloon,
the recessed portions are recessed circumferential portions, and
the recessed circumferential portions extend about a circumference of the balloon.

16. A drug-coated balloon for intracranial atherosclerosis, the drug-coated balloon configured to be inserted through a portion of a main intracranial artery with plaque creating a progressive stenosis, the drug-coated balloon comprising:
a body portion having a distal end, a proximal end opposite the distal end, and a sidewall extending between the distal end and the proximal end, the body portion movable between a deflated position and an inflated position; and
a plurality of wires supported within the body portion, the wires defining concentric rings and longitudinal wires that extend longitudinally along the longitudinal axis at least partially between the distal end and the proximal end, each wire restricting a portion of the balloon from expanding when the body portion moves to the inflated position, the portion of the balloon that is restricted from expanding forms recessed circumferential portions and recessed longitudinal portions in the sidewall of the balloon,
wherein the recessed circumferential portions and the recessed longitudinal portions are configured to reduce an amount of plaque that moves from the main intracranial artery to perforator arteries.

17. The drug-coated balloon of claim 16, wherein:
the longitudinal wires reinforce selected longitudinal portions of the balloon to restrict the selected longitudinal portions of the balloon from expanding,
the wires reinforce selected circumferential portions of the balloon to restrict inflation of the selected circumferential portions of the balloon, and
the recessed portions comprise recessed circumferential portions extending about a circumference of the balloon and recessed longitudinal portions extend at least partially between the distal end and the proximal end.

18. The drug-coated balloon of claim 16, wherein the body portion of the balloon has an elongated oval geometry.

19. The drug-coated balloon of claim 16, wherein:
the longitudinal wires are spaced from each other along an imaginary circle extending through the longitudinal wires, and
the imaginary circle is positioned transverse to the longitudinal axis of the body portion.

20. The drug-coated balloon of claim 19, wherein the wires are equally spaced from each other along a circumference of the imaginary circle.
